(19) United States
(12) Patent Application Publication
(10) Pub. No.: US 2015/0306272 A1
(43) Pub. Date: Oct. 29, 2015

(54) ODOUR-INHIBITING SUPERABSORBER

(71) Applicant: BASF SE, Ludwigshafen (DE)

(72) Inventors: Asif Karim, Mannheim (DE); Thomas Daniel, Waldsee (DE); Christine Schön, Worms (DE)

(73) Assignee: BASF SE, Ludwigshafen (DE)

(21) Appl. No.: 14/651,259

(22) PCT Filed: Dec. 9, 2013
(86) PCT No.: PCT/EP2013/075872

§ 371 Date: Jun. 11, 2015

Related U.S. Application Data
(60) Provisional application No. 61/739,730, filed on Dec. 20, 2012.

(30) Foreign Application Priority Data
Dec. 20, 2012 (EP) 12198318.3

(51) Int. Cl. A61L 15/46; C07C 31/20; A61L 15/20; A61L 15/60; A61L 15/24

(52) U.S. Cl. CPC A61L 15/46; A61L 15/60; A61L 15/24; A61L 15/20; C07C 31/20

(57) ABSTRACT

Superabsorbents comprising 1,2-decanediol exhibit reduced odor formation.

The present invention relates to an odor-inhibiting superabsorbent, to a process for production thereof and to the use thereof, to hygiene articles comprising it and to processes for production thereof.

Superabsorbents are known. For such materials, names such as “highly swellable polymer”, “hydrogel” (often also used for the dry form), “hydrogel-forming polymer”, “water-absorbing polymer”, “absorbent gel-forming material”, “swellable resin”, “water-absorbing resin”, “water-absorbing polymer” or the like are also commonly used. These polymers are crosslinked hydrophilic polymers, more particularly polymers formed from (co)polymerized hydrophilic monomers, graft (co)polymers of one or more hydrophilic monomers on a suitable graft base, crosslinked cellulose ethers or starch ethers, crosslinked carboxymethylcellulose, partly crosslinked polyalkylene oxide or natural products swellable in aqueous liquids, for example guar derivatives, the most common being superabsorbents based on partly neutralized acrylic acid. The essential properties of superabsorbents are their abilities to absorb several times their own weight of aqueous liquids and not to release the liquid again even under a certain pressure. The superabsorbent, which is used in the form of a dry powder, is converted to a gel when it absorbs fluid, and correspondingly to a hydrogel when it absorbs water as usual. Crosslinking is essential for synthetic superabsorbents and is an important difference from customary straightforward thickeners, since it leads to the insolubility of the polymers in water. Soluble substances would be unusable as superabsorbents. By far the most important field of use of superabsorbents is the absorption of body fluids. Superabsorbents are used, for example, in diapers for infants, incontinence products for adults or feminine hygiene products. Other fields of use are, for example, as water-retaining agents in market gardening, as water stores for protection from fire, for fluid absorption in food packaging, or quite generally for absorbing moisture.

Superabsorbents are capable of absorbing several times their own weight of water and of retaining it under a certain pressure. In general, such a superabsorbent has a CRC (“centrifuge retention capacity”, see below for test method) of at least 5 g/g, preferably at least 10 g/g and more preferably at least 15 g/g. A “superabsorbent” may also be a mixture of different individual superabsorbent substances or a mixture of components which exhibit superabsorbent properties only when they interact; it is not so much the substance composition as the superabsorbent properties that are important here.

What is important for a superabsorbent is not just its absorption capacity but also the ability to retain fluid under pressure (retention) and fluid transport in the swollen state. Swollen gel can hinder or prevent fluid transport to as yet unswollen superabsorbent (“gel blocking”). Good transport properties for fluids are possessed, for example, by hydrogels which have a high gel strength in the swollen state. Gels with only a low gel strength are deformable under an applied pressure (body pressure), block pores in the superabsorbent/cellulose fiber suction body and thus prevent further absorption of fluid. An increased gel strength is generally achieved through a higher degree of crosslinking, but this reduces the absorption capacity of the product. An elegant method of increasing the gel strength is that of increasing the degree of crosslinking at the surface of the superabsorbent particles compared to the interior of the particles. To this end, superabsorbent particles which have usually been dried in a surface postcrosslinking step and have an average crosslinking density are subjected to additional crosslinking in a thin surface layer of the particles thereof. The surface postcrosslinking increases the crosslinking density in the shell of the superabsorbent particles, which raises the absorption under compressive stress to a higher level. While the absorption capacity in the surface layer of the superabsorbent particles falls, their core, as a result of the presence of mobile polymer chains, has an improved absorption capacity compared to the shell, such that the shell structure ensures improved onward fluid transport, without occurrence of gel blocking. It is likewise known that superabsorbents which are relatively highly crosslinked overall can be obtained, and that the degree of crosslinking in the interior of the particles can subsequently be reduced compared to an outer shell of the particles.

Processes for producing superabsorbents are also known. Superabsorbents based on acrylic acid, which are the most common on the market, are produced by free-radical polymerization of acrylic acid in the presence of a crosslinker (the “inner crosslinker”), the acrylic acid being neutralized to a certain degree before, after or partly before and partly after the polymerization, typically by adding alkali, usually an aqueous sodium hydroxide solution. The polymer gel thus obtained is comminuted (according to the polymerization reactor used, this can be done simultaneously with the polymerization) and dried. The dry powder thus obtained (the “base polymer”) is typically postcrosslinked on the surface of the particles, by reacting it with further crosslinkers, for instance organic crosslinkers or polyvalent cations, for example aluminum (usually used in the form of aluminum sulfate) or both, in order to obtain a more highly crosslinked surface layer compared to the particle interior.

If superabsorbents are employed in the hygiene sector, they are exposed to body fluids, for example urine or menstrual blood. Such body fluids always comprise unpleasant-smelling components such as amines, fatty acids and other organic components responsible for unpleasant body odors. A further problem with such hygiene products is that the body fluids remain therein over a certain period until the hygiene product is disposed of, and bacterial degradation of nitrogen compounds present in the body fluids to be absorbed, for example urea in urine, gives rise to ammonia or else other amines, which likewise lead to noticeable odor nuisance. Since correspondingly frequent changing of the hygiene product leads to considerable inconvenience, and also cost, to the user or his or her carers, hygiene products which avoid this odor nuisance are advantageous.

There are various known measures for prevention of odor nuisance. Odors can be masked by fragrancing, ammonia or amines formed can be removed by absorption or reaction, and microbial degradation can be inhibited, for example, by biocides or urease inhibitors. These measures can be applied firstly to the superabsorbent and secondly to the hygiene article. Fredric L. Buchholz and Andrew T. Graham (publishers), in: “Modern Superabsorbent Polymer Technology”, J. Wiley & Sons, New York, U.S.A./Wiley-VCH, Weinheim, Germany, 1997, ISBN 0-471-19411-5, give a comprehensive review of superabsorbents, the properties thereof and processes for producing superabsorbents.

EP 1 358 894 A1 teaches hygiene articles which may comprise a series of odor-preventing additives, especially anhydride groups, acid groups, cyclodextrins, biocides, surfactants having an HLB value less than 11, absorbents such as zeolites, clay, activated carbon, silicon dioxide or activated alumina, microorganisms which act as antagonists to unwanted odor-forming microorganisms, pH buffers or chelating agents. In WO 03/076 514 A2, there is a comprehensive overview of the known measures for prevention of unpleasant odors. One measure disclosed is the use of biocides such as Bronopol or glyoxylic acid. In addition, this document teaches a superabsorbent comprising anhydride groups which can react with ammonia or amines and thus bind them in nonvolatile form.

Ammonia and amines are bound at lower pH values than odor-neutral ammonium salts; in addition, a low pH inhibits the growth of ammonia-forming bacteria. EP 202 127 A2 accordingly teaches hygiene articles having a pH buffer, for instance an organic acid or else acid-modified cellulose, which keeps the pH of the skin within the range from 3.0 to 5.5. EP 316 518 A2 relates to superabsorbents composed of partly neutralized polymeric organic acid, optionally additionally with added partly neutralized citric acid, which have a pH between 5.0 and 6.0 in contact with fluid. WO 03/002 623 A1 teaches superabsorbents having a pH below 5.7. GB 2 296 013 A describes hygiene articles comprising a polylactide layer, such that fluid contact gives rise to lactic acid, which lowers the pH. WO 00/35 502 A1 teaches hygiene articles comprising a combination of a buffer having a pH in the range of 3.5-5.5 and lactic acid bacteria. According to WO 00/59 556 A2, a superabsorbent comprising functional groups which can react with ammonia or amines is used, especially cyclic anhydrides, lactides or lactones of hydroxy acids. WO 01/32 226 A1 discloses a superabsorbent with added organic acids.

EP 894 502 A1 teaches the use of cyclodextrins as an absorbent for ammonia in hygiene articles. EP 1 034 800 A1 discloses hygiene articles which, as well as an absorbent for ammonia, especially activated carbon, high-surface area silicon dioxide, clays, zeolites, kieselguhr, chitin, pH buffers, starch, cyclodextrin or ion exchangers, also comprise oxidizing agents such as peracids or diacyl peroxides. WO 91/11 977 A1 relates to zeolites having an SiO2/Al2O3 ratio below 10 as absorbents for odors. According to WO 95/26 207 A1, zeolites having a mean particle size of at least 200 micrometers are used for this purpose.

EP 1 149 597 A1 describes chitosan as an odor-inhibiting component of hygiene articles. EP 1 149 593 A1 teaches cationic polysaccharides, especially chitin derivatives or chitosan, in conjunction with a pH buffer which sets the pH within the range from 3.5 to 6.

EP 739 635 A1 teaches sodium metaborate and tetraborate as urease inhibitors in superabsorbents. According to WO 94/25 077 A1, a mixture of alkali metal or alkaline earth metal tetraborate and boric acid, citric acid, tartaric acid or ascorbic acid as a buffer within the pH range from 7 to 10 is used. WO 03/053486 A1 discloses diapers comprising extract from yucca palms as a urease inhibitor. EP 1 214 878 A1 discloses the use of chelate complexes of divalent metal ions, for instance of the copper complex of monoprotonated ethylenediaminetetraacetate, as a urease inhibitor. WO 95/24173 A2 teaches zeolites impregnated with bactericidal heavy metals such as silver, zinc or copper for odor control.

EP 311 344 A23 relates to hygiene articles which, as well as a pH buffer, comprise a biocide such as alkylammonium halides or bisguanidines. EP 389 023 A2 discloses hygiene articles having an addition selected from biocides and absorbents, especially molecular sieves, for odor control. WO 98/26 808 A2 describes superabsorbents comprising cyclodextrins, zeolites, activated carbon, kieselguhr or acidic salt-forming substances as absorbents for odors, and also biocides, urease inhibitors and pH regulators for inhibition of odor formation.

According to WO 98/20 916 A1, a superabsorbent coated with a biocide such as benzalkonium chloride or chlorhexidine is used in hygiene articles. WO 2007/012 581 A1 relates to storage-stable superabsorbents comprising substituted thiophosphoramides as an odor inhibitor. WO 2009/034 154 A2 and WO 2011/023 647 A1 relate to superabsorbents comprising triclosan. WO 2011/023 560 A2 discloses odor-inhibiting superabsorbents comprising zinc peroxide. The prior patent application having US Patent and Trademark Office reference number 61/657068 discloses superabsorbents comprising flurofamide.

EP 1 269 983 A1 teaches that 1,2-decanediol, applied topically, i.e. applied to the skin, inhibits the growth of microbes which cause body odor on a human or animal body, and thus prevents body odors, especially underarm and foot odor. This document does not teach anything about avoidance of odors as occur in the event of gradual bacterial decomposition of urine or faeces.

The mixing of odor-absorbing high-surface area substances in an adequate amount with superabsorbents in the course of production of hygiene articles lowers the fluid absorption capacity of the mixture. Superabsorbents which themselves have odor-inhibiting properties are those having a low pH. However, these are considerably more difficult to produce than superabsorbents having higher pH values. Less strongly neutralized acrylic acid polymerizes more slowly, and the acidic polymer, in contrast to neutral or more basic polymer, tends to conglutinate, which greatly impairs the necessary further processing (division of the gel, drying, grinding, classifying). In addition, acidic superabsorbents typically have poorer fluid retention capacity under pressure. The use of biocides or antibiotics in hygiene articles, for instance as an addition to the superabsorbent, is disadvantageous since these substances come into contact with the user's skin through diffusion, and act not just against odor-forming bacteria but also in an unwanted manner. In addition, the use thereof, on disposal of the used hygiene articles by the standard route, leads to a considerable introduction of biocides or antibiotics into the environment, which impairs not just the function of water treatment plants, but also contributes to the formation of bacterial strains resistant to antibiotics. Similarly unwanted effects are also associated with the use of bactericidal heavy metals such as zinc, silver or copper.

It is therefore a continuing object to find new, improved or different superabsorbents or superabsorbent-comprising compositions having odor-inhibiting properties or having improved odor inhibition. These should additionally be storage-stable, and should more particularly neither discolor or lose their odor-inhibiting properties even over the course of prolonged storage. Unwanted side effects on skin contact or introduction of constituents into the environment should not occur. In addition, the superabsorbents or compositions should have good fluid absorption and retention properties, especially desirable properties being rapid swelling capacity, good fluid transport properties in the gel bed coupled with simultaneously high absorption capacity, high gel strength and good electrolyte tolerance.

Accordingly, superabsorbents comprising 1,2-decanediol have been found.

When used in a hygiene article, the inventive superabsorbents lead to prevention or at least reduction of unpleasant odors after contact with body fluids. The absorption and retention capacity of the superabsorbent is not significantly impaired by the 1,2-decanediol. It is not necessary, but is possible, to use acidic superabsorbents. The 1,2-decanediol does not impair storage stability; unwanted effects on skin contact or introduction into the environment have neither been observed nor are expected.

In addition, a process for producing the inventive superabsorbents has been found, as have uses of this superabsorbent and hygiene articles comprising this superabsorbent and processes for production thereof.

The inventive superabsorbent comprises generally at least 0.01% by weight of 1,2-decanediol, preferably at least 0.1% by weight and more preferably at least 0.5% by weight of 1,2-decanediol, based in each case on the total weight of the superabsorbent. In general, the inventive superabsorbent comprises at most 10% by weight of 1,2-decanediol, preferably at most 8% by weight and more preferably at most 5% by weight, based in each case on the total weight of the superabsorbent. Examples of contents suitable for most applications are, for instance, 1% by weight or 2% by weight, based in each case on the total weight of the superabsorbent. The upper limit of this proportion by weight is determined by economic rather than technical considerations: the time until occurrence of odors rises with the content of 1,2-decanediol, and it is economically unviable in typical applications of superabsorbents, especially in hygiene products such as diapers, to extend this time beyond the typical wearing time of such hygiene products.

The superabsorbent preferably comprises 1,2-n-decanediol.

For the rest, i.e. apart from the content of 1,2-decanediol, the inventive superabsorbent is a customary superabsorbent which, more particularly, aside from 1,2-decanediol, may comprise all other known additions, constituents or assistants.

The inventive superabsorbent has preferably been surface postcrosslinked.

The process according to the invention for production of the inventive superabsorbents differs from known polymerization processes for production of superabsorbents merely in that 1,2-decanediol is added to the superabsorbent. In other words, any known process for producing superabsorbents can be practiced in the manner of the invention by addition of 1,2-decanediol.

In the simplest manner, 1,2-decanediol is added to an existing superabsorbent. For this purpose, the superabsorbent can simply be mixed with 1,2-decanediol. “Masterbatch” methodology can likewise be used for this purpose, i.e. a superabsorbent not comprising any 1,2-decanediol can be mixed with a superabsorbent comprising a much higher amount of 1,2-decanediol than desired in the end product, in such a way that the finished mixture comprises the desired amount of 1,2-decanediol.

1,2-Decanediol can be added in solid form, as a melt or in suspension or solution. Preferably, 1,2-decanediol is added as a solution; the solvent used may be any solvent in which the amount of 1,2-decanediol to be applied dissolves. Suitable examples are alcohols, for instance methanol, ethanol, n- and isopropanol, n-, iso- and sec-butanol.

Preferably, 1,2-decanediol is added after drying or, if surface postcrosslinking is effected, after the surface postcrosslinking. If other process steps associated with a heat treatment of the superabsorbent are performed, the 1,2-decanediol is preferably added after the last heat treatment. This avoids the possibility, which would otherwise exist, of loss of 1,2-decanediol owing to the effect, which is possible in principle, of diols as surface postcrosslinkers. A convenient and preferred method of applying the 1,2-decanediol as a melt to the superabsorbent may exist if the superabsorbent, after a heat treatment, still has a temperature above the melting point of 1,2-decanediol but below a temperature at which significant reaction takes place between acid groups of the superabsorbent and 1,2-decanediol. In general, a suitable temperature of the superabsorbent for this purpose is at least 50° C., preferably at least 55° C. and more preferably at least 60° C., and at most 150° C., preferably at most 120° C. and more preferably at most 100° C.

A preferred polymerization process according to the invention for production of acrylate superabsorbents comprising 1,2-decanediol is the aqueous solution polymerization of a monomer mixture comprising
a) at least one ethylenically unsaturated monomer which bears acid groups and is optionally present at least partly in salt form,
b) at least one crosslinker,
c) at least one initiator,
d) optionally one or more ethylenically unsaturated monomers copolymerizable with the monomers mentioned under a), and
e) optionally one or more water-soluble polymers,
   drying the polymer thus obtained, optionally grinding, optionally classifying and optionally surface postcrosslinking, and admixing with 1,2-decanediol.

The monomers a) are preferably water-soluble, i.e. the solubility in water at 23° C. is typically at least 1 g/100 g of water, preferably at least 5 g/100 g of water, more preferably at least 25 g/100 g of water and most preferably at least 35 g/100 g of water.

Suitable monomers a) are, for example, ethylenically unsaturated carboxylic acids or salts thereof, such as acrylic acid, methacrylic acid, maleic acid, maleic anhydride and itaconic acid or salts thereof. Particularly preferred monomers are acrylic acid and methacrylic acid. Very particular preference is given to acrylic acid.

Further suitable monomers a) are, for example, ethylenically unsaturated sulfonic acids, such as styrenesulfonic acid and 2-acrylamido-2-methylpropanesulfonic acid (AMPS).

Impurities can have a considerable influence on the polymerization. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, an acrylic acid purified according to WO 2004/035514 A1 and comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The proportion of acrylic acid and/or salts thereof in the total amount of monomers a) is preferably at least 50 mol %, more preferably at least 90 mol %, most preferably at least 95 mol %.

The monomer solution comprises preferably at most 250 ppm by weight, preferably at most 130 ppm by weight, more preferably at most 70 ppm by weight and preferably at least 10 ppm by weight, more preferably at least 30 ppm by weight, especially around 50 ppm by weight, of hydroquinone monoether, based in each case on the unneutralized monomer a); neutralized monomer a), i.e. a salt of the monomer a), is considered for arithmetic purposes to be unneutralized monomer. For example, the monomer solution can be prepared by using an ethylenically unsaturated monomer bearing acid groups with an appropriate content of hydroquinone monoether.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerized free-radically into the polymer chain, and functional groups which can form covalent bonds with the acid groups of the monomer a). In addition, polyvalent metal salts which can form coordinate bonds with at least two acid groups of the monomer a) are also suitable as crosslinkers b).

Crosslinkers b) are preferably compounds having at least two polymerizable groups which can be polymerized free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 530 438 A1, di- and triacrylates, as described in EP 547 847 A1, EP 559 476 A1, EP 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/32962 A2.

Preferred crosslinkers b) are pentaerythrityl triallyl ether, tetraallyloxyethane, methylenebismethacrylamide, 10 to 20-tuply ethoxylated trimethylolpropane triacrylate, 10 to 20-tuply ethoxylated trimethylolethane triacrylate, more preferably 15-tuply ethoxylated trimethylolpropane triacrylate, polyethylene glycol diacrylates having 4 to 30 ethylene oxide units in the polyethylene glycol chain, trimethylolpropane triacrylate, di- and triacrylates of 3 to 30-tuply ethoxylated glycerol, more preferably di- and triacrylates of 10-20-tuply ethoxylated glycerol, and triallylamine. The polyols incompletely esterified with acrylic acid may also be present here as Michael adducts with one another, as a result of which it is also possible for tetraacrylates, pentaacrylates or even higher acrylates to be present.

Very particularly preferred crosslinkers b) are the polyethoxylated and/or -propoxylated glycerols which have been esterified with acrylic acid or methacrylic acid to give di- or triacrylates, as described, for example, in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably from 0.05 to 1.5% by weight, more preferably from 0.1 to 1% by weight, most preferably from 0.3 to 0.6% by weight, based in each case on monomer a). With rising crosslinker content, the centrifuge retention capacity (CRC) falls and the absorption under a pressure of 0.3 psi (AUL0.3psi) rises.

The initiators c) used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is, however, preferably a mixture of the sodium salt of 2-hydroxy-2-sulfinatoacetic acid, the disodium salt of 2-hydroxy-2-sulfonatoacetic acid and sodium bisulfite (in the form of Bruggolit® FF6M or Bruggolit® FF7, or alternatively BRUGGOLITE® FF6M or BRUGGOLITE® FF7, available from L. Bruggemann K G, Salzstrasse 131, 74076 Heilbronn, Germany, www.brueggemann.com).

Ethylenically unsaturated monomers d) copolymerizable with the ethylenically unsaturated monomers a) bearing acid groups are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate, maleic acid and maleic anhydride.

The water-soluble polymers e) used may be polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

Typically, an aqueous monomer solution is used. The water content of the monomer solution is preferably from 40 to 75% by weight, more preferably from 45 to 70% by weight and most preferably from 50 to 65% by weight. It is also possible to use monomer suspensions, i.e. oversaturated monomer solutions. With rising water content, the energy requirement in the subsequent drying rises, and, with falling water content, the heat of polymerization can only be removed inadequately.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

The monomer mixture may comprise further components. Examples of further components used in monomer mixtures of this kind are, for instance, chelating agents, in order to keep metal ions in solution. In addition, all other additives to the monomer mixture which are known in superabsorbent production can be used.

Suitable polymerization reactors are, for example, kneading reactors or belt reactors. In the kneader, the polymer gel formed in the polymerization of an aqueous monomer solution or suspension is comminuted continuously by, for example, contrarotatory stirrer shafts, as described in WO 2001/38402 A1. Polymerization on a belt is described, for example, in DE 38 25 366 A1 and U.S. Pat. No. 6,241,928. Polymerization in a belt reactor forms a polymer gel, which has to be comminuted in a further process step, for example in a meat grinder, extruder or kneader. It is also possible to produce spherical superabsorbent particles by suspension, spray or droplet polymerization processes.

The acid groups of the resulting polymer gels have typically been partially neutralized. Neutralization is preferably carried out at the monomer stage; in other words, salts of the monomers bearing acid groups or, to be precise, a mixture of monomers bearing acid groups and salts of the monomers bearing acid groups (“partly neutralized acid”) are used as component a) in the polymerization. This is typically accomplished by mixing the neutralizing agent as an aqueous solution or preferably also as a solid into the monomer mixture intended for polymerization or preferably into the monomer bearing acid groups or a solution thereof. The degree of neutralization is preferably from 25 to 95 mol %, more preferably from 50 to 80 mol % and most preferably from 65 to 72 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metal cations are sodium and potassium, but very particular preference is given to sodium hydroxide, sodium carbonate or sodium hydrogencarbonate and also mixtures thereof.

However, it is also possible to carry out neutralization after the polymerization, at the stage of the polymer gel formed in the polymerization. It is also possible to neutralize up to 40 mol %, preferably 10 to 30 mol % and more preferably 15 to 25 mol % of the acid groups before the polymerization by adding a portion of the neutralizing agent directly to the monomer solution and setting the desired final degree of neutralization only after the polymerization, at the polymer gel stage. When the polymer gel is neutralized at least partly after the polymerization, the polymer gel is preferably comminuted mechanically, for example by means of an extruder, in which case the neutralizing agent can be sprayed, sprinkled or poured on and then carefully mixed in. To this end, the gel mass obtained can be repeatedly extruded for homogenization.

However, preference is given to performing the neutralization at the monomer stage. In other words: in a very particularly preferred embodiment, the monomer a) used is a mixture of 25 to 95 mol %, more preferably from 50 to 80 mol % and most preferably from 65 to 72 mol % of salt of the monomer bearing acid groups, and the remainder to 100 mol % of monomer bearing acid groups. This mixture is, for example, a mixture of sodium acrylate and acrylic acid or a mixture of potassium acrylate and acrylic acid.

In a preferred embodiment, the neutralizing agent used for the neutralization is one whose iron content is generally below 10 ppm by weight, preferably below 2 ppm by weight and more preferably below 1 ppm by weight. Likewise desired is a low content of chloride and anions of oxygen acids of chlorine. A suitable neutralizing agent is, for example, the 50% by weight sodium hydroxide solution or potassium hydroxide solution which is typically traded as “membrane grade”; even more pure and preferred, but also more expensive, is the 50% by weight sodium hydroxide solution or potassium hydroxide solution typically traded as “amalgam grade” or “mercury process”.

The polymer gel obtained from the aqueous solution polymerization and optional subsequent neutralization is then preferably dried with a belt drier until the residual moisture content is preferably 0.5 to 15% by weight, more preferably 1 to 10% by weight and most preferably 2 to 8% by weight (see below for test method for the residual moisture or water content). In the case of too high a residual moisture content, the dried polymer gel has too low a glass transition temperature Tg and can be processed further only with difficulty. In the case of too low a residual moisture content, the dried polymer gel is too brittle and, in the subsequent comminution steps, undesirably large amounts of polymer particles with an excessively low particle size are obtained (“fines”). The solids content of the gel before drying is generally from 25 to 90% by weight, preferably from 30 to 80% by weight, more preferably from 35 to 70% by weight and most preferably from 40 to 60% by weight. Optionally, however, it is also possible to dry using a fluidized bed drier or a heatable mixer with a mechanical mixing unit, for example a paddle drier or a similar drier with mixing tools of different design. Optionally, the drier can be operated under nitrogen or another nonoxidizing inert gas or at least under reduced partial oxygen pressure in order to prevent oxidative yellowing processes. In general, however, even sufficient venting and removal of water vapor leads to an acceptable product. In general, a minimum drying time is advantageous with regard to color and product quality. In the case of the standard belt driers, in a customary mode of operation, a temperature of the gas used for drying of at least 50° C., preferably at least 80° C. and more preferably at least 100° C., and generally of at most 250° C., preferably at most 200° C. and more preferably of at most 180° C. is established for this purpose. Standard belt driers often have two or more chambers; the temperature in these chambers may be different. For each drier type, the operating conditions overall should be chosen in a known manner such that the desired drying outcome is achieved.

During the drying, the residual monomer content in the polymer particles is also reduced, and last residues of the initiator are destroyed.

In some polymerization processes, for instance spray or droplet polymerization, polymerization and drying take place essentially simultaneously, i.e. a dry product is obtained directly at the outlet of the polymerization reactor and no separate drying step is conducted, or is conducted merely optionally for further reduction of the water content in the product.

The dried polymer gel is subsequently optionally ground and optionally classified. In some polymerization processes, for instance suspension, spray or droplet polymerization, the desired particle size distributions of the superabsorbent can be established as early as the polymerization step. If this is done, grinding and classification can be dispensed with, or serve merely for removal of an unwanted fraction of excessively large or excessively small particles.

Grinding can be accomplished using all known mills, for instance one- or multistage roll mills, preferably two or three-stage roll mills, pinned disk mills, hammer mills or vibratory mills. Oversize gel lumps which often still have not dried on the inside are elastomeric, lead to problems in the grinding and are preferably removed before the grinding, which can be done in a simple manner by wind sifting or by means of a sieve (“guard sieve” for the mill). In view of the mill used, the mesh size of the sieve should be selected such that a minimum level of disruption resulting from oversize, elastomeric particles occurs.

Excessively large superabsorbent particles are perceptible as coarse particles in their predominant use, in hygiene products such as diapers; they also lower the mean initial swell rate of the superabsorbent. Both are undesirable. Advantageously, coarse-grain polymer particles are therefore removed from the product. This is done by conventional classification processes, for example wind sifting, or by sieving through a sieve with a mesh size of at most 1000 μm, preferably at most 900 μm, more preferably at most 850 μm and most preferably at most 800 μm. For example, sieves of mesh size 700 μm, 650 μm or 600 μm are used. The coarse polymer particles (“oversize”) removed may, for cost optimization, be sent back to the grinding and sieving cycle or be processed further separately.

Polymer particles with too small a particle size lower the permeability (SFC). Advantageously, this classification therefore also removes fine polymer particles. This can, if sieving is effected, conveniently be effected through a sieve of mesh size at most 300 μm, preferably at most 200 μm, more preferably at most 150 μm and most preferably at most 100 μm. The fine polymer particles (“undersize” or “fines”) removed can, for cost optimization, be sent back as desired to the monomer stream, to the polymerizing gel, or to the fully polymerized gel before the drying of the gel.

The mean particle size of the polymer particles obtained as the product fraction is generally at least 200 μm, preferably at least 250 μm and more preferably at least 300 μm, and generally at most 600 μm and more preferably at most 500 μm. The proportion of particles with a particle size of at least 150 μm is generally at least 90% by weight, more preferably at least 95% by weight and most preferably at least 98% by weight. The proportion of particles with a particle size of at most 850 μm is generally at least 90% by weight, more preferably at least 95% by weight and most preferably at least 98% by weight.

The polymer thus prepared has superabsorbent properties and is covered by the term “superabsorbent”. Its CRC is typically comparatively high, but its AUL or SFC comparatively low. A surface nonpostcrosslinked superabsorbent of this type is often referred to as “base polymer” to distinguish it from a surface postcrosslinked superabsorbent produced therefrom.

To further improve the properties, especially increase the AUL and SFC values (which usually lowers the CRC value), the superabsorbent particles are optionally surface postcrosslinked. Suitable postcrosslinkers are compounds which comprise groups which can form bonds with at least two functional groups of the superabsorbent particles. In the case of the acrylic acid/sodium acrylate-based superabsorbents prevalent on the market, suitable surface postcrosslinkers are compounds which comprise groups which can form bonds with at least two carboxylate groups. Postcrosslinkers and processes for postcrosslinking are known; it is possible to use any known postcrosslinker and any known postcrosslinking process.

Examples of postcrosslinkers are 2-oxazolidone, N-methyl-2-oxazolidone, N-(2-hydroxyethyl)-2-oxazolidone and N-hydroxypropyl-2-oxazolidone; 1,3-propanediol, 1,5-pentanediol, 1,6-hexanediol and 1,7-heptanediol, 1,3-butanediol, 1,8-octanediol, 1,9-nonanediol and 1,10-decanediol; butane-1,2,3-triol, butane-1,2,4-triol, glycerol, trimethylolpropane, trimethylolethane, pentaerythritol, 1- to 3-tuply ethoxylated glycerol, trimethylolethane or trimethylolpropane (per molecule), and 1- to 3-tuply propoxylated glycerol, trimethylolethane or trimethylolpropane (per molecule); 2-tuply ethoxylated or propoxylated neopentyl glycol; ethylene carbonate and propylene carbonate; 2,2′-bis(2-oxazoline); or else mixtures of two or more of these compounds.

The postcrosslinker is generally used in an amount of at least 0.001% by weight, preferably of at least 0.02% by weight, more preferably of at least 0.05% by weight, and generally at most 2% by weight, preferably at most 1% by weight, more preferably at most 0.3% by weight, for example at most 0.15% by weight or at most 0.095% by weight, based in each case on the mass of the base polymer.

The postcrosslinking is typically performed in such a way that a solution of the postcrosslinker is sprayed onto the dried base polymer particles. After the spray application, the polymer particles coated with postcrosslinker are dried thermally, and the postcrosslinking reaction can take place either before or during the drying. The spray application of the postcrosslinker solution is preferably carried out in mixers with moving mixing tools, such as screw mixers, disk mixers, paddle mixers or shovel mixers, or mixers with other mixing tools. Particular preference is given, however, to vertical mixers. It is also possible to spray on the postcrosslinker solution in a fluidized bed. Suitable mixers are obtainable, for example, as Pflugschar® plowshare mixers from Gebr. Lodige Maschinenbau GmbH, Elsener-Strasse 7-9, 33102 Paderborn, Germany, or as Schugi® Flexomix® mixers, Vrieco-Nauta® mixers or Turbulizer® mixers from Hosokawa Micron BV, Gildenstraat 26, 7000 AB Doetinchem, the Netherlands.

The postcrosslinkers are typically used in the form of an aqueous solution. If exclusively water is used as the solvent, a surfactant or deagglomeration assistant is advantageously added to the postcrosslinker solution or actually to the base polymer. This improves the wetting characteristics and reduces the tendency to form lumps. The aqueous postcrosslinker solution may, as well as the at least one postcrosslinker, also comprise a cosolvent. The content of nonaqueous solvent or total amount of solvent can be used to adjust the penetration depth of the postcrosslinker into the polymer particles. Industrially highly suitable cosolvents are C1-C6-alcohols such as methanol, ethanol, n-propanol, isopropanol, n-butanol, sec-butanol, tert-butanol or 2-methyl-1-propanol, C2-05-diols such as ethylene glycol, 1,2-propylene glycol or 1,4-butanediol, ketones such as acetone, or carboxylic esters such as ethyl acetate.

The concentration of the at least one postcrosslinker in the aqueous postcrosslinker solution is typically from 1 to 20% by weight, preferably from 1.5 to 10% by weight and more preferably from 2 to 5% by weight, based on the postcrosslinker solution.

The total amount of the postcrosslinker solution based on base polymer is typically from 0.3 to 15% by weight and preferably from 2 to 6% by weight.

The actual surface postcrosslinking by reaction of the surface postcrosslinker with functional groups at the surface of the base polymer particles is usually carried out by heating the base polymer wetted with surface postcrosslinker solution, typically referred to as “drying” (but not to be confused with the above-described drying of the polymer gel from the polymerization, in which typically very much more liquid has to be removed). The drying can be effected in the mixer itself, by heating the jacket, by means of heat exchange surfaces or by blowing in warm gases. Simultaneous admixing of the superabsorbent with surface postcrosslinker and drying can be effected, for example, in a fluidized bed drier. The drying is, however, usually carried out in a downstream drier, for example a tray drier, a rotary tube oven, a paddle or disk drier or a heatable screw. Suitable driers are obtainable, for example, as Solidair® or Torusdisc® driers from Bepex International LLC, 333 N.E. Taft Street, Minneapolis, Minn. 55413, U.S.A., or as paddle or shovel driers or else as fluidized bed driers from Nara Machinery Co., Ltd., European office, Europaallee 46, 50226 Frechen, Germany.

Preferred drying temperatures are in the range of 100 to 250° C., preferably 120 to 220° C., more preferably 130 to 210° C. and most preferably 150 to 200° C. The preferred residence time at this temperature in the reaction mixer or drier is preferably at least 10 minutes, more preferably at least 20 minutes, most preferably at least 30 minutes, and typically at most 60 minutes. Typically, the drying is conducted such that the superabsorbent has a residual moisture content of generally at least 0.1% by weight, preferably at least 0.2% by weight and most preferably at least 0.5% by weight, and generally at most 15% by weight, preferably at most 10% by weight and more preferably at most 8% by weight.

Before, during or after the postcrosslinking, polyvalent cations can be applied to the particle surface in addition to the postcrosslinkers. This is in principle a further surface postcrosslinking by means of ionic noncovalent bonds, but is occasionally also referred to as “complexation” with the metal ions in question or simply as “coating” with the substances in question (the “complexing agent”). Metal ions for complexation, compounds of such metal ions and processes for application are known; it is possible to use any known metal ion, any known compound thereof and any known process for application.

This application of polyvalent cations is typically effected by spray application of solutions of di- or polyvalent cations, usually di-, tri- or tetravalent metal cations, but also polyvalent cations such as polymers formed, in a formal sense, entirely or partly from vinylamine monomers, such as partly or fully hydrolyzed polyvinylamide (so-called “polyvinylamine”), whose amine groups are always—even at very high pH values—present partly in protonated form to give ammonium groups. Examples of usable divalent metal cations are especially the divalent cations of metals of groups 2 (especially Mg, Ca, Sr, Ba), 7 (especially Mn), 8 (especially Fe), 9 (especially Co), 10 (especially Ni), 11 (especially Cu) and 12 (especially Zn) of the Periodic Table of the Elements. Examples of usable trivalent metal cations are especially the trivalent cations of metals of groups 3 including the lanthanides (especially Sc, Y, La, Ce), 8 (especially Fe), 11 (especially Au), 13 (especially Al) and 14 (especially Bi) of the Periodic Table of the Elements. Examples of usable tetravalent cations are especially the tetravalent cations of metals from the lanthanides (especially Ce) and group 4 (especially Ti, Zr, Hf) of the Periodic Table of the Elements. The metal cations can be used either alone or as a mixture with one another. Particular preference is given to the use of trivalent metal cations. Very particular preference is given to the use of aluminum cations.

Among the metal cations mentioned, suitable metal salts are all of those which possess sufficient solubility in the solvent to be used. Particularly suitable metal salts are those with weakly complexing anions, for example chloride, nitrate and sulfate, hydrogensulfate, carbonate, hydrogencarbonate, nitrate, phosphate, hydrogenphosphate, or dihydrogenphosphate. Preference is given to salts of mono- and dicarboxylic acids, hydroxy acids, keto acids and amino acids, or basic salts. Preferred examples include acetates, propionates, tartrates, maleates, citrates, lactates, malates, succinates. Likewise preferred is the use of hydroxides. Particular preference is given to the use of 2-hydroxycarboxylic salts such as citrates and lactates. Examples of particularly preferred metal salts are alkali metal and alkaline earth metal aluminates and hydrates thereof, for instance sodium aluminate and hydrates thereof, alkali metal and alkaline earth metal lactates and citrates and hydrates thereof, aluminum acetate, aluminum propionate, aluminum citrate and aluminum lactate.

The cations and salts mentioned can be used in pure form or as a mixture of different cations or salts. The salts of the di- and/or trivalent metal cation used may comprise further secondary constituents such as still unneutralized carboxylic acid and/or alkali metal salts of the neutralized carboxylic acid. Preferred alkali metal salts are those of sodium and potassium, and those of ammonium. They are typically used in the form of an aqueous solution which is obtained by dissolving the solid salts in water, or is preferably obtained directly as such, which avoids any drying and purification steps. It is advantageously also possible to use the hydrates of the salts mentioned, which often dissolve more rapidly in water than the anhydrous salts.

The amount of metal salt used is generally at least 0.001% by weight, preferably at least 0.01% by weight and more preferably at least 0.1% by weight, for example at least 0.4% by weight, and generally at most 5% by weight, preferably at most 2.5% by weight and more preferably at most 1% by weight, for example at most 0.7% by weight, based in each case on the mass of the base polymer.

The salt of the trivalent metal cation can be used in the form of a solution or suspension. Solvents used for the metal salts may be water, alcohols, DMF, DMSO and mixtures of these components. Particular preference is given to water and water/alcohol mixtures, for example water/methanol, water/1,2-propanediol and water/1,3-propanediol.

The treatment of the base polymer with solution of a di- or polyvalent cation is effected in the same manner as that with surface postcrosslinker, including the drying step. Surface postcrosslinker and polyvalent cation can be sprayed on in a combined solution or as separate solutions. The spray application of the metal salt solution to the superabsorbent particles may either precede or follow the surface postcrosslinking. In a particularly preferred process, the spray application of the metal salt solution is effected in the same step together with the spray application of the crosslinker solution, in which case the two solutions are sprayed on separately in succession or simultaneously via two nozzles, or crosslinker solution and metal salt solution can be sprayed on jointly via one nozzle.

It is also possible to add all further additives known in the surface postcrosslinking of superabsorbents. Examples are basic salts of a divalent metal cation such as calcium or strontium, usually in the form of hydroxides, hydrogencarbonates, carbonates, acetates, propionates, citrates, gluconates, lactates, tartrates, malates, succinates, maleates and/or fumarates. Further examples are reducing compounds such as hypophosphites, phosphonic acid derivatives, sulfinates or sulfites.

If a drying step is carried out after the surface postcrosslinking and/or treatment with complexing agent, it is advantageous but not absolutely necessary to cool the product after the drying. The cooling can be effected continuously or batchwise; to this end, the product is conveniently conveyed continuously into a cooler arranged downstream of the drier. Any apparatus known for removal of heat from pulverulent solids can be used for this purpose, more particularly any device mentioned above as drying apparatus, except that it is charged not with a heating medium but with a cooling medium, for example with cooling water, such that no heat is introduced into the superabsorbent via the walls and, according to the construction, also via the stirring elements or other heat exchange surfaces, and is instead removed therefrom. Preference is given to the use of coolers in which the product is moved, i.e. cooled mixers, for example shovel coolers, disk coolers or paddle coolers. The superabsorbent can also be cooled in a fluidized bed by injecting a cooled gas such as cold air. The cooling conditions are adjusted so as to obtain a superabsorbent with the temperature desired for further processing. Typically, a mean residence time in the cooler of generally at least 1 minute, preferably at least 3 minutes and more preferably at least 5 minutes, and generally at most 6 hours, preferably at most 2 hours and more preferably at most 1 hour is established, and the cooling performance is such that the product obtained has a temperature of generally at least 0° C., preferably at least 10° C. and more preferably at least 20° C., and generally at most 100° C., preferably at most 80° C. and more preferably at most 60° C.

As described above, a convenient way of adding 1,2-decanediol may be to add it at a product temperature at which it does not react significantly with superabsorbent but has melted. The abovementioned temperature range, which is suitable in most cases, is typically passed through in the cooler, and so the addition of 1,2-decanediol in the cooler is a convenient and preferred mode of addition. In the case of standard continuous coolers with intensive product mixing, it may be sufficient to add 1,2-decanediol in substance at a point in the cooler where the superabsorbent has the chosen suitable temperature, meaning that 1,2-decanediol melts and is mixed, but does not react significantly with the superabsorbent. It is of course also possible to add a melt, suspension or solution in the cooler.

The surface postcrosslinked superabsorbent is optionally ground and/or sieved in a customary manner. Grinding is typically not required here, but the removal by sieving of agglomerates or fines formed is usually appropriate for establishment of the desired particle size distribution of the product. Agglomerates and fines are either discarded or preferably recycled into the process in a known manner at a suitable point, agglomerates after comminution. The particle sizes desired for surface postcrosslinked superabsorbents are the same as for base polymers.

It is optionally possible to additionally apply to the surface of the superabsorbent particles, whether unpostcrosslinked or postcrosslinked, in any process step of the production process, if required, all known coatings such as film-forming polymers, thermoplastic polymers, dendrimers, polycationic polymers (for example polyvinylamine, polyethyleneimine or polyallylamine), water-insoluble polyvalent metal salts, for example magnesium carbonate, magnesium oxide, magnesium hydroxide, calcium carbonate, calcium sulfate or calcium phosphate, all water-soluble mono- or polyvalent metal salts known to those skilled in the art, for example aluminum sulfate, sodium salts, potassium salts, zirconium salts or iron salts, or hydrophilic inorganic particles such as clay minerals, fumed silica, colloidal silica sols, for example Levasil®, titanium dioxide, aluminum oxide and magnesium oxide. Examples of useful alkali metal salts are sodium and potassium sulfate, and sodium and potassium lactates, citrates and sorbates. This makes it possible to achieve additional effects, for example a reduced caking tendency of the end product or of the intermediate in the particular process step of the production process, improved processing properties or a further enhanced saline flow conductivity (SFC). When the additives are used and sprayed on in the form of dispersions, they are preferably used in the form of aqueous dispersions, and preference is given to additionally applying an antidusting agent to fix the additive on the surface of the superabsorbent. The antidusting agent is then either added directly to the dispersion of the inorganic pulverulent additive; optionally, it can also be added as a separate solution before, during or after the application of the inorganic pulverulent additive by spray application. Most preferred is the simultaneous spray application of postcrosslinking agent, antidusting agent and pulverulent inorganic additive in the postcrosslinking step. In a further preferred process variant, the antidusting agent is, however, added separately in the cooler, for example by spray application from above, below or from the side. Particularly suitable antidusting agents which can also serve to fix pulverulent inorganic additives on the surface of the superabsorbent particles are polyethylene glycols with a molecular weight of 400 to 20 000 g/mol, polyglycerol, 3- to 100-tuply ethoxylated polyols, such as trimethylolpropane, glycerol, sorbitol and neopentyl glycol. Particularly suitable are 7- to 20-tuply ethoxylated glycerol or trimethylolpropane, for example Polyol TP 70® (Perstorp, Sweden). The latter have the advantage, more particularly, that they lower the surface tension of an aqueous extract of the superabsorbent particles only insignificantly.

It is likewise possible to adjust the inventive superabsorbent to a desired water content by adding water.

Optionally, the inventive superabsorbents are provided with further additives which stabilize against discoloration. Examples are especially known stabilizers against discoloration, especially reducing substances. Among these, preference is given to solid or dissolved salts of phosphinic acid (H₃PO₂) and to this acid itself. For example, all phosphinates of the alkali metals are suitable, including those of ammonium, and of the alkaline earth metals. Particular preference is given to aqueous solutions of phosphinic acid which comprise phosphinate ions and at least one cation selected from sodium, potassium, ammonium, calcium, strontium, aluminum, magnesium. Preference is likewise given to salts of phosphonic acid (H₃PO₃) and to the latter itself. For example, all primary and secondary phosphonates of the alkali metals, including those of ammonium, and of the alkaline earth metals, are suitable. Particular preference is given to aqueous solutions of phosphonic acid which comprise primary and/or secondary phosphonate ions and at least one cation selected from sodium, potassium, calcium, strontium.

All coatings, solids, additives and assistants can each be added in separate process steps, but the most convenient method is usually to add them—if they are not added during the admixing of the base polymer with surface postcrosslinking agent—to the superabsorbent in the cooler, for instance by spray application of a solution or addition in fine solid form or in liquid form.

The inventive superabsorbent generally has a centrifuge retention capacity (CRC) of at least 5 g/g, preferably of at least 10 g/g and more preferably of at least 20 g/g. Further suitable minimum CRC values are, for example, 25 g/g, 30 g/g or 35 g/g. It is typically not more than 40 g/g. A typical CRC range for surface postcrosslinked superabsorbents is from 28 to 33 g/g.

If it has been surface postcrosslinked, the inventive superabsorbent typically has an absorption under load (AUL0.7psi, for test method see below) of at least 18 g/g, preferably at least 20 g/g, more preferably at least 22 g/g, especially preferably at least 23 g/g, most preferably at least 24 g/g, and typically not more than 30 g/g.

The inventive superabsorbent additionally has a saline flow conductivity (SFC, see below for test method) of at least 10×10⁻⁷cm³s/g, preferably at least 30×10⁻⁷cm³s/g, more preferably at least 50×10⁻⁷cm³s/g, especially preferably at least 80×10⁻⁷cm³s/g, most preferably at least 100×10⁻⁷cm³s/g, and typically not more than 1000×10⁻⁷cm³s/g.

The present invention further provides hygiene articles comprising inventive superabsorbent, preferably ultrathin diapers, comprising an absorbent layer consisting of 50 to 100% by weight, preferably 60 to 100% by weight, more preferably 70 to 100% by weight, especially preferably 80 to 100% by weight and very especially preferably 90 to 100% by weight of inventive superabsorbent, of course not including the envelope of the absorbent layer.

Very particularly advantageously, the inventive superabsorbents are also suitable for production of laminates and composite structures, as described, for example, in US 2003/0181115 and US 2004/0019342. In addition to the hotmelt adhesives described in both documents for production of such novel absorbent structures, and especially the fibers, described in US 2003/0181115, composed of hotmelt adhesives to which the superabsorbent particles are bound, the inventive superabsorbents are also suitable for production of entirely analogous structures using UV-crosslinkable hotmelt adhesives, which are sold, for example, as AC-Resin® (BASF SE, Carl-Bosch-Strasse 38, 67056 Ludwigshafen, Germany). These UV-crosslinkable hotmelt adhesives have the advantage of already being processable at 120 to 140° C.; they therefore have better compatibility with many thermoplastic substrates. A further significant advantage is that UV-crosslinkable hotmelt adhesives are very benign in toxicological terms and also do not cause any vaporization in the hygiene articles. A very significant advantage in connection with the inventive superabsorbents is the property of the UV-crosslinkable hotmelt adhesives of lacking any tendency to yellow during processing and crosslinking. This is especially advantageous when ultrathin or partly transparent hygiene articles are to be produced. The combination of the inventive superabsorbents with UV-crosslinkable hotmelt adhesives is therefore particularly advantageous. Suitable UV-crosslinkable hotmelt adhesives are described, for example, in EP 0 377 199 A2, EP 0 445 641 A1, U.S. Pat. No. 5,026,806, EP 0 655 465 A1 and EP 0 377 191 A2.

The inventive superabsorbent can also be used in other fields of industry in which fluids or liquids, especially water or aqueous solutions, are absorbed. These fields are, for example, storage, packaging, transport (as constituents of packaging material for water- or moisture-sensitive articles, for instance for flower transport, and also as protection against mechanical effects); animal hygiene (in cat litter); food packaging (transport of fish, fresh meat; absorption of water, blood in fresh fish or meat packaging); medicine (wound plasters, water-absorbing material for burn dressings or for other weeping wounds), cosmetics (carrier material for pharmaceutical chemicals and medicaments, rheumatic plasters, ultrasonic gel, cooling gel, cosmetic thickeners, sunscreen); thickeners for oil/water or water/oil emulsions; textiles (moisture regulation in textiles, shoe insoles, for evaporative cooling, for instance in protective clothing, gloves, headbands); chemical engineering applications (as a catalyst for organic reactions, for immobilization of large functional molecules such as enzymes, as an adhesive in agglomerations, heat stores, filtration aids, hydrophilic components in polymer laminates, dispersants, liquefiers); as assistants in powder injection molding, in the building and construction industry (installation, in loam-based renders, as a vibration-inhibiting medium, assistants in tunnel excavations in water-rich ground, cable sheathing); water treatment, waste treatment, water removal (deicers, reusable sand bags); cleaning; agrochemical industry (irrigation, retention of melt water and dew deposits, composting additive, protection of forests from fungal/insect infestation, retarded release of active ingredients to plants); for firefighting or for fire protection; coextrusion agents in thermoplastic polymers (for example for hydrophilization of multilayer films); production of films and thermoplastic moldings which can absorb water (e.g. films which store rain and dew for agriculture; films comprising superabsorbents for maintaining freshness of fruit and vegetables which are packaged in moist films; superabsorbent-polystyrene coextrudates, for example for packaging foods such as meat, fish, poultry, fruit and vegetables); or as a carrier substance in active ingredient formulations (pharmaceuticals, crop protection).

The inventive articles for absorption of fluid differ from known examples in that they comprise the inventive superabsorbent.

A process for producing articles for absorption of fluid, especially hygiene articles, has also been found, said process comprising using at least one inventive superabsorbent in the production of the article in question. In addition, processes for producing such articles using superabsorbents are known.

### Test Methods

The superabsorbent is tested by the test methods described below.

The standard test methods described hereinafter and designated “WSP” are described in: “Standard Test Methods for the Nonwovens Industry”, 2005 edition, published jointly by the Worldwide Strategic Partners EDANA (European Disposables and Nonwovens Association, Avenue Eugene Plasky, 157, 1030 Brussels, Belgium, www.edana.org) and INDA (Association of the Nonwoven Fabrics Industry, 1100 Crescent Green, Suite 115, Cary, N.C. 27518, U.S.A., www.inda.org). This publication is available both from EDANA and from INDA.

All measurements described below should, unless stated otherwise, be conducted at an ambient temperature of 23±2° C. and a relative air humidity of 50±10%. The superabsorbent particles are mixed thoroughly before the measurement unless stated otherwise.

Centrifuge Retention Capacity (CRC)

The centrifuge retention capacity of the superabsorbent is determined by the standard test method No. WSP 241.5-02 “Centrifuge retention capacity”.

Absorbency Under a Load of 0.3 psi (AUL0.3 psi)

The absorbency under a load of 2068 Pa (0.3 psi) of the superabsorbent is determined by the standard test method No. WSP 242.2-05 “Absorption under pressure”.

Absorbency Under a Load of 0.7 psi (AUL0.7 psi)

The absorbency under a load of 4826 Pa (0.7 psi) of the superabsorbent is determined analogously to the standard test method No. WSP 242.2-05 “Absorption under pressure”, except using a weight of 49 g/cm² (leads to a load of 0.7 psi) instead of a weight of 21 g/cm² (leads to a load of 0.3 psi).

Moisture Content of the Superabsorbent (Residual Moisture, Water Content)

The water content of the superabsorbent particles is determined by the standard test method No. WSP 230.2-05 “Moisture content”.

Mean Particle Size

The mean particle size of the product fraction is determined by the standard test method No. WSP 220.2-05 “Particle size distribution”.

Determination of odor inhibition:

To assess the odor-inhibiting effect of the inventive compositions, the inhibition of the bacterial formation of ammonia from urea is determined. DSM1 medium (Deutsche Sammlung von Mikroorganismen and Zellkulturen GmbH) was prepared from 5.0 g/l meat peptone (Merck KGaA; Darmstadt; Germany; Art. No. 1.07214) and 3.0 g/l meat extract (Merck KGaA; Darmstadt; Germany; Art. No. 1103979) and adjusted to pH =7.0. 50 ml of DSM1 medium were inoculated with Proteus mirabilis ATCC 14153 to OD=0.1 and incubated in a 250 ml baffled Erlenmeyer flask at 37° C. and 220 rpm for 15 hours. The cultures thus produced had a cell density of about 109 CFU/ml (OD=2.0−2.5).

Synthetic urine was produced from 25 g/l urea (sterile-filtered), 9.0 g/l sodium chloride, 1 g/l meat peptone and 1 g/l meat extract. The synthetic urine was autoclaved prior to addition of the sterile-filtered concentrated urea solution.

125 ml polypropylene histology cups were autoclaved and charged with the amount of superabsorbent particles needed for absorption of 50 ml of synthetic urine (calculated from the centrifuge retention capacity). Then 50 ml of synthetic urine were inoculated with 50 μL of bacterial strain solution corresponding to an overall concentration of about 106 CFU/ml and mixed with the superabsorbent particles, and the lid, provided with a diffusion test tube (Drägerwerk A G & Co. KGaA; Lübeck; DE; Dräger Tube® ammonia 20/a-D; Article No. 8101301), was screwed on immediately. The evolution of ammonia was observed at 37° C. over 48 hours.

The measurement noted was the time of first occurrence of ammonia detectable by the test tube.

### EXAMPLES

### Example 1

A laboratory mixer (manufacturer: Waring Products, Inc., Torrington, Conn., U.S.A., model 34 BL 99 (8012)) having two opposite rounded mixing paddles and baffles on the lid (comparable results are also achieved in many other mixers with good mixing during the introduction of the solution, though it should be ensured that the stirrer units do not comminute the superabsorbent—the stirrer speed should be set accordingly) was initially charged with 20 g of Hysorb® B 7055 superabsorbent (BASF SE, Ludwigshafen, Germany). A disposable syringe was used to add 0.968 g of a previously prepared mixture of 1.6494 g of isopropanol and 0.4296 g of 1,2-decanediol (from TCI Europe N.V. Boerenveldseweg 6—Haven 1063, 2070 Zwijndrecht, Belgium, product number D2720, CAS 1119-86-4) dropwise to the mixed polymer at moderate stirrer level (level 3 of 7) of the mixer. Subsequently, the mixer was opened briefly, in order to push the contents together in the middle with a brush. This was followed by brief mixing, again at level 3, and then the product was emptied into a Petri dish which was placed in a fume hood for 45 minutes. A free-flowing product was obtained.

The CRC of the product thus obtained was 30.9 g/g. In the odor inhibition test, ammonia first occurred after 21 hours.

### Example 2

Example 1 was repeated. The CRC of the product thus obtained was 30.9 g/g. In the odor inhibition test, ammonia first occurred after 16 hours.

### Example 3 (Comparative)

The Hysorb® B 7055 superabsorbent which had not been admixed with 1,2-decanediol had a CRC of 30.4 g/g. In the odor inhibition test, ammonia first occurred after 8.2 hours.

### Example 4 (Comparative)

Example 1 was repeated, except that, rather than the solution of 1,2-decanediol, 1.00 g of a previously prepared solution of 0.4148 g of 1,2-octanediol (from Alfa Aesar GmbH & Co KG, Zeppelinstr. 7b, 76185 Karlsruhe, Germany, product number L08031, CAS 1117-86-8) in 1.9792 g of isopropanol was added dropwise.

The CRC of the product thus obtained was 31.1 g/g. In the odor inhibition test, ammonia first occurred after 10 hours.

### Example 5 (Comparative)

Example 4 was repeated. The CRC of the product thus obtained was 31.1 g/g. In the odor inhibition test, ammonia first occurred after 10 hours.

### Example 6 (Comparative)

Example 1 was repeated, except that 30 g rather than 20 g of superabsorbent were used and, rather than the solution of 1,2-decanediol, 1.5 g of a previously prepared solution of 1 g of 1,2-dodecanediol (from Sigma-Aldrich Chemie GmbH, Riedstraβe 2, 89555 Steinheim, Germany, product number 213721, CAS 1119-87-5) in 4 g of isopropanol was added dropwise. The CRC of the product thus obtained was 31.0 g/g. In the odor inhibition test, ammonia first occurred after 10 hours.

The comparison of the examples with the comparative examples shows that the addition of 1,2-decanediol to the superabsorbent can reduce or prevent the formation of ammonia and hence of unpleasant odors over typical periods of diaper use, without impairing the absorption properties.

1. A superabsorbent comprising 1,2-decanediol.

2. The superabsorbent according to claim 1, which comprises 1,2-decanediol in an amount of 0.01 to 10% by weight, based on the total weight of the superabsorbent.

3. The superabsorbent according to claim 2, which has been surface postcrosslinked.

4. A process for producing a superabsorbent defined in claim 1 comprising adding 1,2-decanediol to superabsorbent.

5. The process according to claim 4, which comprises polymerizing an aqueous solution of a monomer mixture comprising:
a) at least one ethylenically unsaturated monomer which bears an acid group and is optionally present at least partly in salt form,
b) at least one crosslinker,
c) at least one initiator,
d) optionally one or more ethylenically unsaturated monomer copolymerizable with the monomer mentioned under a),
e) optionally one or more water-soluble polymer, drying, optionally grinding, optionally classifying, and optionally surface postcrosslinking the polymer thus obtained, and admixing it with 1,2-decanediol.

6. An article for absorption of fluids, comprising a superabsorbent defined in claim 1.

7. (canceled)

8. An article for absorption of a fluid comprising a superabsorbent defined in claim 1.

9. The superabsorbent of claim 1 comprising a crosslinked polymer of an ethylenically unsaturated carboxylic acid, an ethylenically unsaturated sufonic acid, or salt thereof.
